(19) United States
(12) Patent Application Publication
(10) Pub. No.: US 2023/0149690 A1
(43) Pub. Date: May 18, 2023

(54) BYPASS SHEATH

(71) Applicant: Boston Scientific Scimed Inc., Maple Grove, MN (US)

(72) Inventors: Qian Liu, Plymouth, MN (US); Brian R. Reynolds, Ramsey, MN (US)

(21) Appl. No.: 17/989,155

(22) Filed: Nov. 17, 2022

Related U.S. Application Data
(60) Provisional application No. 63/280,446, filed on Nov. 17, 2021.

(51) Int. Cl. A61M 60/122; A61M 60/857

(52) U.S. Cl. CPC A61M 60/122; A61M 60/857; A61M 60/414

(57) ABSTRACT

An introducer sheath for use in delivering devices includes a proximal portion opposite a distal portion and a body portion extending between the proximal portion and the distal portion, the body portion defining a lumen of the introducer sheath configured for receiving at least one device, at least one bypass opening positioned in the proximal portion of the introducer sheath such that the bypass opening extends from an exterior of the introducer sheath to the lumen of the introducer sheath, and at least one plug configured to positioned within the lumen of the introducer sheath and to inhibit the flow of blood into the lumen proximal of the plug.

### CROSS REFERENCE TO RELATED APPLICATION

This application claims priority to Provisional Application No. 63/280,446, filed Nov. 17, 2021, which is herein incorporated by reference in its entirety.

### TECHNICAL FIELD

The present disclosure relates to the field of introducer sheaths for use in the delivery of percutaneous devices. Specifically, the present disclosure relates to a bypass sheath for use in the intravascular delivery of percutaneous devices.

### BACKGROUND

In various procedures, for example procedures for delivering percutaneous medical devices, an introducer sheath is introducer into a blood vessel of a patient, for example a femoral artery, and the medical devices to be inserted through the blood vessel are inserted into the introducer sheath for introduction into the blood vessel. To provide sufficient volume for introducing the devices, the introducer sheath may have the same, or almost the same, diameter as the blood vessel. As such, the introducer sheath may substantially reduce or essentially block the blood flow through the blood vessel while the introducer sheath is placed within the artery, which can cause various detrimental effects on the patient.

### SUMMARY

In Example 1, an introducer sheath for use in delivering devices includes a proximal portion opposite a distal portion and a body portion extending between the proximal portion and the distal portion, the body portion defining a lumen of the introducer sheath configured for receiving at least one device, at least one bypass opening positioned in the proximal portion of the introducer sheath such that the bypass opening extends from an exterior of the introducer sheath to the lumen of the introducer sheath, and at least one plug positioned within the lumen of the introducer sheath and configured to inhibit the flow of blood into the lumen proximal of the plug.

In Example 2, the introducer sheath of Example 1 further includes wherein the at least one bypass opening includes a plurality of bypass openings arranged at the proximal portion of the introducer sheath.

In Example 3, the introducer sheath of Example 1 further includes wherein the at least one plug is positioned directly adjacent the at least one bypass opening in a direction proximal to the at least one bypass opening.

In Example 4, the introducer sheath of Example 2 further includes wherein the at least one plug is positioned directly adjacent a proximal most bypass opening of the plurality of bypass openings.

In Example 5, the introducer sheath of Example 1 further includes wherein the at least one plug is composed of silicone.

In Example 6, the introducer sheath of Example 1 further includes wherein the at least one plug has a radiopaque coating.

In Example 7, the introducer sheath of Example 1 further includes wherein the at least one plug is composed of a radiopaque material.

In Example 8, the introducer sheath of Example 1 further includes wherein the at least one bypass opening is lined by a radiopaque material.

In Example 9, the introducer sheath of Example 1 further includes wherein the at least one plug comprises a central opening for sealingly engaging with a catheter positioned within the introducer sheath.

In Example 10, the introducer sheath of Example 9 further includes wherein the central opening is composed of a plurality of slits.

In Example 11, a method for delivering a device into a patient includes inserting an introducer sheath into a vessel of the patient, the introducer sheath including a proximal portion opposite a distal portion, a body portion extending between the proximal portion and the distal portion and the body portion defining a lumen, and at least one bypass opening arranged at the proximal portion of the introducer sheath. The method further includes inserting the device into the introducer sheath and advancing the device to a target position in the patient and inserting a plug into the introducer sheath arranged such that the plug is positioned adjacent to and proximally relative to the at least one bypass opening to inhibit the flow of blood into the lumen proximal of the plug.

In Example 12, the method of Example 11 further includes wherein removing the plug from the introducer sheath and removing the percutaneous circulatory support device through the introducer sheath.

In Example 13, the method of Example 11 further includes wherein the at least one bypass opening includes a plurality of bypass openings and the plug is positioned proximally of a proximal-most bypass opening of the plurality of bypass openings.

In Example 14, the method of Example 11 further includes wherein the at least one plug having a central opening for sealingly engaging with a catheter positioned within the introducer sheath.

In Example 15, the method of Example 14 further includes wherein the central opening is composed of a plurality of slits.

In Example 16, an introducer sheath for use in delivering percutaneous devices, the introducer sheath including a proximal portion opposite a distal portion and a body portion extending between the proximal portion and the distal portion, the body portion defining a lumen of the introducer sheath configured for receiving at least one percutaneous device, at least one bypass opening positioned in the proximal portion of the introducer sheath such that the bypass opening extends from an exterior of the introducer sheath to the lumen of the introducer sheath, and at least one plug positioned within the lumen of the introducer sheath and configured to inhibit the flow of blood into the lumen proximal of the plug.

In Example 17, the introducer sheath of Example 16 further includes wherein the at least one bypass opening includes a plurality of bypass openings arranged at the proximal portion of the introducer sheath.

In Example 18, the introducer sheath of Example 16 further includes wherein the at least one plug is positioned directly adjacent the at least one bypass opening in a direction proximal to the at least one bypass opening.

In Example 19, the introducer sheath of Example 17 further includes wherein the at least one plug is positioned directly adjacent a proximal-most bypass opening of the plurality of bypass openings.

In Example 20, the introducer sheath of Example 16 further includes wherein the at least one plug is composed of silicone.

In Example 21, the introducer sheath of Example 16 further includes wherein the at least one plug has a radiopaque coating.

In Example 22, the introducer sheath of Example 16 further includes wherein the at least one plug is composed of a radiopaque material.

In Example 23, the introducer sheath of Example 16 further includes wherein the at least one bypass opening is lined by a radiopaque material.

In Example 24, the introducer sheath of Example 16 further includes wherein the at least one plug includes a central opening for sealingly engaging with a catheter positioned within the introducer sheath.

In Example 25, the introducer sheath of Example 24 further includes wherein the central opening is composed of a plurality of slits.

In Example 26, a percutaneous device delivery system for delivering a percutaneous device includes a percutaneous device having an impeller housing for supporting an impeller and a motor configured to rotatably drive the impeller. The system further includes an introducer sheath for receiving the percutaneous device, the introducer sheath having a proximal portion, a distal portion, and a body portion extending between the proximal portion and the distal portion, the body portion defining a lumen, wherein the proximal portion comprises at least one bypass opening extending from an exterior of the introducer sheath to the lumen of the introducer sheath, and wherein the introducer sheath further includes a plug positioned within the lumen and proximally relative to the at least one bypass opening of the introducer sheath to inhibit the flow of blood into the lumen proximal of the plug.

In Example 27, the system of Example 26 further includes wherein the at least one bypass opening is lined with a radiopaque material.

In Example 28, the system of Example 26 further includes wherein the plug is composed of a radiopaque material.

In Example 29, the system of Example 26 further includes wherein the at least one bypass opening includes a plurality of bypass openings.

In Example 30, the system of Example 26 further includes wherein the at least one bypass opening includes a plurality of bypass openings.

In Example 31, the system of Example 26 further includes wherein the plug includes a central opening configured for receiving a catheter positioned within the introducer sheath.

In Example 32, the system of Example 26 further includes wherein the plug includes a central opening composed of a slit.

In Example 33 a method for delivering a percutaneous device into a patient includes inserting an introducer sheath into a vessel of the patient, the introducer sheath including a proximal portion opposite a distal portion, a body portion extending between the proximal portion and the distal portion and the body portion defining a lumen, and at least one bypass opening arranged at the proximal portion of the introducer sheath. The method further includes inserting the percutaneous device into the introducer sheath and advancing the device to a target position in the patient and inserting a plug into the introducer sheath arranged such that the plug is positioned adjacent to and proximally relative to the at least one bypass opening to inhibit the flow of blood into the lumen proximal of the plug.

In Example 34, the method of Example 33 further includes wherein the method further includes removing the plug from the introducer sheath and removing the percutaneous circulatory support device through the introducer sheath.

In Example 35, the method of Example 33 further includes wherein the at least one bypass opening includes a plurality of bypass openings and the plug is positioned proximally of a proximal-most bypass opening of the plurality of bypass openings.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a side view of an introducer sheath after insertion into a blood vessel, in accordance with embodiments of the present disclosure.

FIG. 2 illustrates a side cross-sectional view of the introducer sheath of FIG. 1 after insertion into the blood vessel, in accordance with embodiments of the present disclosure.

FIG. 3 illustrates an additional side cross-sectional view of a variation of the introducer sheath of FIG. 1 after insertion into the blood vessel, in accordance with embodiments of the present disclosure.

FIG. 4A illustrates a top cross sectional view of an embodiment of a plug for the introducer sheath of FIG. 1, in accordance with embodiments of the present disclosure.

FIG. 4B illustrates a top cross sectional view of a variation of the plug for the introducer sheath of FIG. 4A, in accordance with embodiments of the present disclosure.

FIG. 5 illustrates a sectional side cutaway figure showing the introducer sheath of FIG. 1 after receiving a percutaneous circulatory support device and insertion into the blood vessel.

FIG. 6 illustrates a method for deploying a delivery device through the introducer sheath of FIG. 1, in accordance with embodiments of the present disclosure.

### DETAILED DESCRIPTION

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

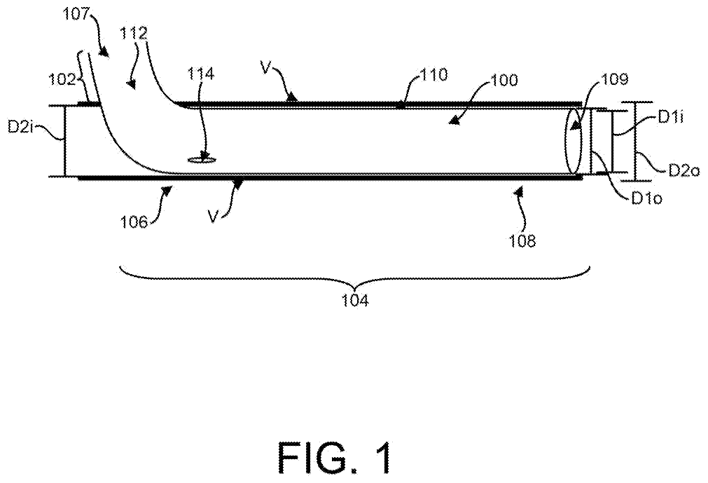

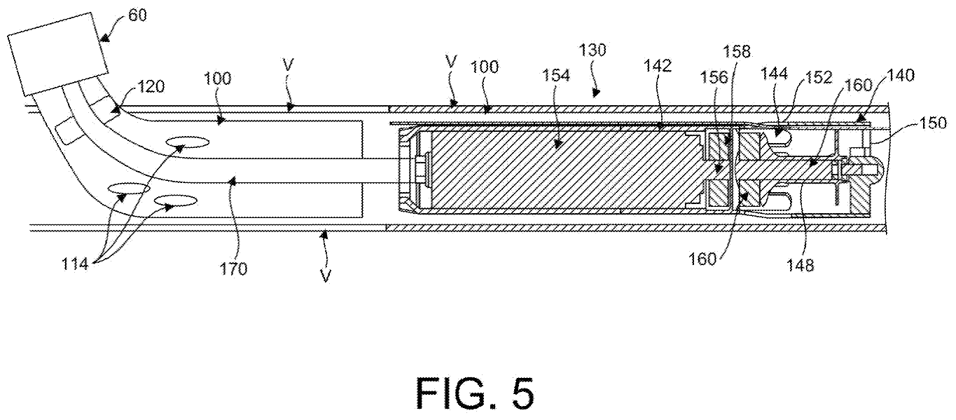

FIG. 1 illustrates a side cross sectional view of a blood vessel V with an introducer sheath 100, also referred to herein as the bypass sheath 100, inserted at least partially into the blood vessel V. The introducer sheath 100 comprises an outer portion 102 that is positioned outside of the blood vessel V and an inner portion 104 that is positioned within the blood vessel V, or circumferentially enveloped by the blood vessel V. Specifically, with reference to the inner portion 104 of the introducer sheath 100, the inner portion 104 comprises a proximal portion 106 and a distal portion 108 that is opposite the proximal portion 106. The introducer sheath 100 includes a proximal opening 107 adjacent the proximal portion 106 and a distal opening 109 adjacent the distal portion 108. A body portion 110 of the introducer sheath 100 extends between the proximal portion 106 and the distal portion 108, and the body portion 110 defines a lumen 112 of the introducer sheath 100. The introducer sheath 100 may be formed by various polymeric or metallic materials. In further embodiments, the introducer sheath 100 may comprise an additional surface coating. The surface coating may include, but is not limited to, silicone, PET, or any other applicable polymer. A hub 60 (FIG. 5) is commonly included at the proximal opening 107. The hub 60 may contain a valve (not shown) for hemostasis, i.e, to prevent blood from leaking out of the introducer sheath during use.

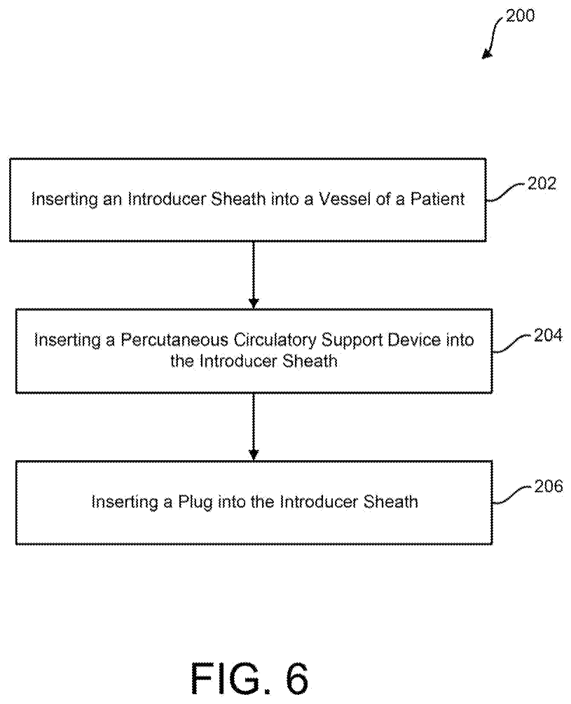

In the illustrative embodiment of FIG. 1, the lumen 112 is cylindrical in shape, however various other configurations may be incorporated. Additionally, the body portion 110 of the introducer sheath 100 comprises an inner diameter D1i defining the lumen 112 and an outer diameter D1o. The blood vessel V is defined by an inner diameter D2i and an outer diameter D2o. In embodiments, the outer diameter D1o is slightly less than the inner diameter D2i of the blood vessel V. For example, the outer diameter D1o may be 95% of the value of the inner diameter D2i of the blood vessel V. In various other embodiments, the outer diameter D1o may be between 96% and 99% of the inner diameter D2i of the blood vessel V. The inner diameter D1i and outer diameter D1o of the introducer sheath 100 and lumen 112 may be configured this way to allow for a device to pass through the introducer sheath 100, as will be described further with reference to FIGS. 5-6. In this way, any spacing between the outer diameter D1o of the introducer sheath 100 and the inner diameter D2i of the blood vessel V is reduced. As such, any blood flow between the introducer sheath 100 and the blood vessel V is minimized or potentially essentially blocked. This may cause various complications to a patient, as the blood is not able to properly flow to the rest of the body. In order to allow for continued blood flow, at least one bypass opening may be provided within the introducer sheath 100, as will be described further herein.

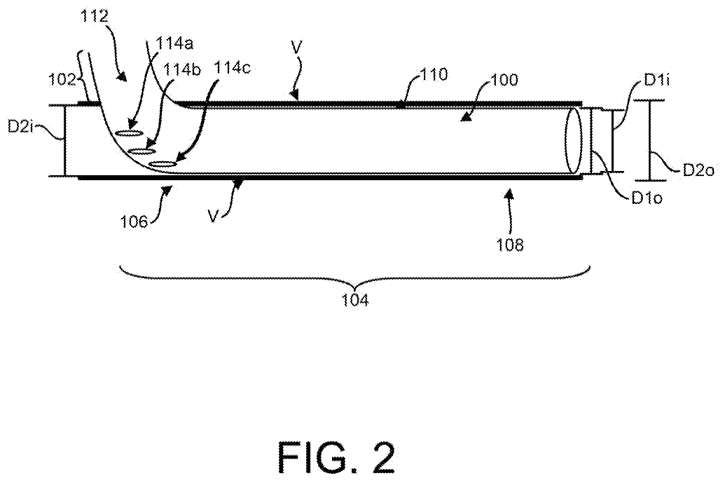

With continued reference to the introducer sheath 100 of FIG. 1, the proximal portion 106 includes at least one bypass opening 114 extending through the body portion 110. In other words, the at least one bypass opening 114 extends from an exterior of the body portion 110 through to the lumen 112 of the introducer sheath 100. In the illustrative embodiment of the FIG. 2, the introducer sheath 100 comprises a plurality of bypass openings and more specifically, a first bypass opening 114a, a second bypass opening 114b, and a third bypass opening 114c. The first bypass opening 114a is a proximal-most bypass opening of the proximal portion 106 and the third bypass opening 114c is a distal-most bypass opening of the proximal portion 106. While illustrated as three bypass openings, the plurality of bypass openings may include any number of bypass openings. For example, the plurality of bypass openings may include four, five, six or more bypass openings. Additionally, the distribution and the positioning of the plurality of bypass openings may vary from the example presented herein. For example, the plurality of bypass openings may be positioned aligned with one another in a horizontal or a vertical direction. The plurality of bypass openings may be clustered with one another and/or the plurality of bypass openings may be positioned randomly along the body portion 110. Further, the plurality of bypass openings may be aligned such that the bypass openings 114 are aligned with anatomical features, such as branching blood vessels. The plurality of bypass openings may be varied in size, positioning, and/or shape in order to optimize blood flow through the introducer sheath 100.

The configuration of the introducer sheath 100 and the bypass opening 114 allows for increased blood flow through blood vessel V as compared with traditional introducer sheaths by allowing blood to flow through the introducer sheath 100. In traditional introducer sheaths without a bypass opening, blood that flows into the introducer sheath is substantially inhibited from flowing past the introducer sheath in a blood vessel. Thus, blood flow in the blood vessel may be essentially blocked at the location of the traditional introducer sheath. In contrast, blood flowing into the distal portion 108 of introducer sheath 100 as described herein is able to exit at the proximal portion 106 of the introducer sheath 100 due to the presence of the at least one bypass opening 114. This allows the blood entering at the distal portion 108 to flow through the introducer sheath 100 and back into blood vessel V via the bypass opening 114, thereby reducing the potential of the blood flow ceasing in the introducer sheath 100. Such an arrangement also contributes to continued blood flow through the vessel V past introducer sheath 100, which may be particularly advantageous when the outer diameter D1o of the introducer sheath 100 is slightly less than the inner diameter D2i of the blood vessel V. As previously described, in these instances, there is a very small space, or no space, for blood to flow between the introducer sheath 100 and the blood vessel V.

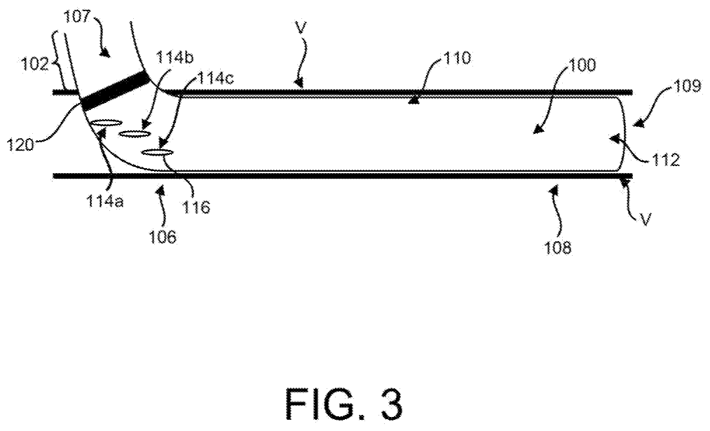

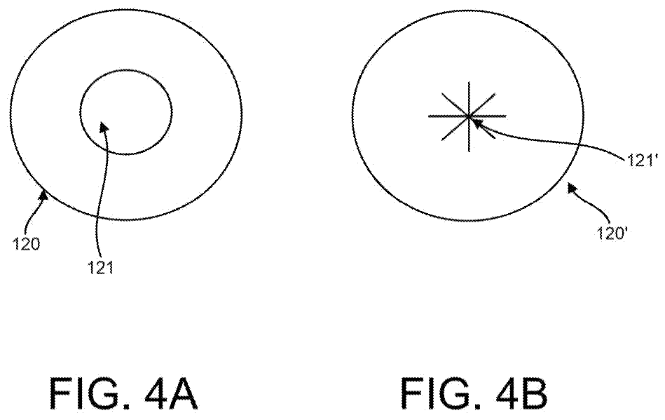

In some embodiments, it is advantageous to incorporate a plug within the introducer sheath 100 in order to reduce the potential for the accumulation of stagnant blood within the introducer sheath 100 in a portion that is proximal to the at least one bypass opening. Specifically, as will be described further with reference to FIGS. 4 and 5, the plug may be used after a percutaneous circulatory support device is delivered through the introducer sheath 100. For example, FIG. 3 illustrates the introducer sheath 100 of FIG. 1 with the addition of at least one plug within the introducer sheath 100. Specifically, the introducer sheath 100 includes a plug 120. The plug 120 is illustrated positioned just proximally of the plurality of bypass openings, and is positioned adjacent the proximal-most bypass opening 114a of the introducer sheath 100. In this way, the blood may proximally flow through the introducer sheath 100, first entering the distal portion 108 of the introducer sheath 100 and then out of the sheath 100 through the plurality of bypass openings 114a-c at the proximal portion 106. Because the plug 120 is positioned proximal to the plurality of bypass openings 114a-c, the blood is prevented or inhibited from flowing within introducer sheath 100 into a portion of the lumen 112 of the introducer sheath 100 that is proximal to the plurality of bypass openings 114 the blood would be unable to exit the introducer sheath 100. Thus, accumulation of blood in the introducer sheath 100 proximally of the plurality of bypass openings 114a-c is substantially reduced.

The plug 120 may have a cylindrical shape with one or more bypass openings to accommodate a catheter 170 (FIG. 5) or various other tools that are inserted into the introducer sheath 100. In these embodiments, the plug 120 may form a fluid tight seal between the plug 120 and the catheter 170 and/or tools, and the plug 120 and the introducer sheath 100 to ensure blood flow is prohibited. For example, an opening in the center of plug 120 may accommodate the catheter 170 (FIG. 5) of percutaneous circulatory support device 130, as shown in FIGS. 4A-5. The opening of the plug 120 may take a variety of forms, including a slotted opening, cross slits, or any other configuration that allows the plug 120 to be coupled to the catheter 170 without accessing the ends of the catheter 170. For example, FIG. 4A illustrates the plug 120 having a circular shape with a circular opening 121 within the center of the plug 120. In use, the circular opening 121 is sized to sealingly engage with the catheter 170 once placed over the catheter 170. FIG. 4B illustrates a variation of the plug, illustratively the plug 120′, which has an overall circular shape and cross slits 121′ within the center of the plug 120′ that allow for the plug 120′ to be placed over the catheter 170 and form a fluid tight seal between the catheter 170, the plug 120, and the introducer sheath 100. However, various other configurations of the opening of the plugs 120, 120′ may be incorporated and the illustrative embodiments herein are only presented as examples.

Further, it may be beneficial for the physician to have markers along the introducer sheath 100 for indicating the positioning of the introducer sheath 100 and the plurality of bypass openings 114a-c. For example, with reference to FIG. 2, the plurality of bypass openings 114a-c may each have an outer lining (or a circumferential lining) 116 that is composed of radiopaque material. This provides the additional advantage to the physician of being able to detect the positioning of the plurality of bypass openings 114a-c, and thus the inner portion 104 of the introducer sheath 100, when it is placed within the blood vessel V of the patient. In further embodiments, the plug 120 may be composed of a material including, but not limited to, silicone. In these embodiments, the plug 120 may have a radiopaque coating such that the physician is able to detect the positioning of the plug 120 within the inner portion 104. In other embodiments, the plug 120 may be formed entirely of a radiopaque material. The positioning of either the plurality of bypass openings 114a-c or the plug 120 may thus be detected through the use of imaging tests, such as an ultrasound, X-ray, and/or angiogram. For example, the radiopaque material may be selected such that the plurality of bypass openings 114a-114c may be identified through the use of these various tests. This allows the physician to ensure that the plurality of bypass openings 114a-c are in the desired position and orientation, and that the plug 120 is positioned proximally to the plurality of bypass openings 114a-c.

As previously described, the introducer sheath 100 of FIGS. 1 and 2 are used for delivery of a medical device, for example a percutaneous circulatory support device, as will be described further with reference to FIG. 5. While described with reference to the percutaneous circulatory support device of FIG. 5, the introducer sheath 100 may be used with any variation of a medical device, and the following description is provided as an example.

FIG. 5 illustrates an example of the introducer sheath 100 in use with a percutaneous circulatory support device 130, also referred to herein as a blood pump 130. Specifically, FIG. 5 illustrates the introducer sheath 100 just after the blood pump 130 has exited the distal portion 108 of the introducer sheath 100 and is located in the blood vessel V. The blood pump 130 is thus positioned distally relative to the introducer sheath 100 and the introducer sheath 100 maintains positioning within the blood vessel V. The blood pump 130 generally includes an impeller assembly housing 140 and a motor housing 142. In some embodiments, the impeller assembly housing 140 and the motor housing 142 may be integrally or monolithically constructed. In other embodiments, the impeller assembly housing 140 and the motor housing 142 may be separate components configured to be removably or permanently coupled.

The impeller assembly housing 140 carries an impeller assembly 144 therein. The impeller assembly 144 includes an impeller 148 that rotates relative to the impeller assembly housing 140 to drive blood through the blood pump 130. More specifically, the impeller 148 causes blood to flow from a blood inlet 150 formed on the impeller assembly housing 140, through the impeller assembly housing 140, and out of a blood outlet 152 formed on the impeller assembly housing 140. The inlet 150 may be formed on an end portion of the impeller assembly housing 140 and the outlet 152 may be formed on a side portion of the impeller assembly housing 140. In other embodiments, the inlet 150 and/or the outlet 152 may be formed on other portions of the impeller assembly housing 140. In some embodiments, the impeller assembly housing 140 may couple to a distally extending cannula (not shown), and the cannula may receive and deliver blood to the inlet 150.

With continued reference to FIG. 5, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. In other embodiments, the motor 154 may couple to the impeller assembly housing 140 via other components.

As shown in FIG. 5, the catheter 170 extends from the proximal portion of motor housing 142. The catheter 170 typically contains tools such as control wires and in some examples, a guidewire, for delivering medical devices to the target location and allowing for operation of the medical devices. The tools may also be used for removing the percutaneous circulatory support device 130 from the patient. The catheter 170 extends through introducer sheath 100, optionally through plug 120, and outside the patient's body. FIG. 5 illustrates that blood may flow through introducer sheath 100 due to the plurality of openings 114a-c, as the blood can pass through the percutaneous circulatory support device 130 and into the distal portion 108 of the introducer sheath 100 and out the plurality of bypass openings 114a-c.

FIG. 6 illustrates a flow chart disclosing a method 200 of using the introducer sheath 100 as illustrated in FIGS. 1-3 and 5 in combination with the plug 120 as disclosed with reference to FIGS. 3-4B. At block 202, the method 200 includes inserting the introducer sheath 100 into the blood vessel V of the patient. This may include inserting the introducer sheath 100 into an incision site of the patient such that at least inner portion 104 extends within the blood vessel V. At block 204, the method 200 further includes inserting the percutaneous circulatory support device 130 into the introducer sheath 100. In various embodiments, this includes first introducing the plug 120 into a starter tube, inserting the starter tube into the introducer sheath 100 and then deploying the plug 120 into the introducer sheath 100.

At block 206, the method 200 further includes inserting the plug 120 into the introducer sheath 100. This step may further comprise inserting the plug 120 such that the plug 120 is flush with an inner surface of the lumen 112 of the introducer sheath 100 and an outer surface of a catheter or starter tube such that blood is unable to pass by or through the plug 120. The plug 120 may be positioned proximally to the proximalmost bypass opening 114a of the introducer sheath 100 such that blood flow through each of the plurality of bypass openings 114 is not impeded.

In various embodiments, the method 200 further includes removing the plug 120 from the introducer sheath 100 such that the plug 120 may then be removed from the introducer sheath 100, as well. As previously disclosed, while the introducer sheath 100 is described with reference to use with plug 120, any variety of medical devices may be delivered through the introducer sheath 100 as described herein.

One further advantage of the introducer sheath 100 as described herein is that it eliminates the need for a separate repositioning sheath to be incorporated into the system. That is, the introducer sheath 100 may remain in place the entire time that the percutaneous circulatory support device 130 is in the patient. Removing the introducer sheath 100 and positioning an additional sheath is not necessary which may reduce the instances of traumatic injury or stress onto the blood vessel V.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

1. An introducer sheath for use in delivering percutaneous devices, the introducer sheath comprising:
a proximal portion opposite a distal portion and a body portion extending between the proximal portion and the distal portion, the body portion defining a lumen of the introducer sheath configured for receiving at least one percutaneous device;
at least one bypass opening positioned in the proximal portion of the introducer sheath such that the bypass opening extends from an exterior of the introducer sheath to the lumen of the introducer sheath; and
at least one plug positioned within the lumen of the introducer sheath and configured to inhibit the flow of blood into the lumen proximal of the plug.

2. The introducer sheath of claim 1, wherein the at least one bypass opening comprises a plurality of bypass openings arranged at the proximal portion of the introducer sheath.

3. The introducer sheath of claim 1, wherein the at least one plug is positioned directly adjacent the at least one bypass opening in a direction proximal to the at least one bypass opening.

4. The introducer sheath of claim 2, wherein the at least one plug is positioned directly adjacent a proximal-most bypass opening of the plurality of bypass openings.

5. The introducer sheath of claim 1, wherein the at least one plug is composed of silicone.

6. The introducer sheath of claim 1, wherein the at least one plug has a radiopaque coating.

7. The introducer sheath of claim 1, wherein the at least one plug is composed of a radiopaque material.

8. The introducer sheath of claim 1, wherein the at least one bypass opening is lined by a radiopaque material.

9. The introducer sheath of claim 1, wherein the at least one plug comprises a central opening for sealingly engaging with a catheter positioned within the introducer sheath.

10. The introducer sheath of claim 9, wherein the central opening is composed of a plurality of slits.

11. A percutaneous device delivery system for delivering a percutaneous device, the system comprising:
a percutaneous device comprising
an impeller housing for supporting an impeller,
a motor configured to rotatably drive the impeller;
an introducer sheath for receiving the percutaneous device, the introducer sheath having a proximal portion, a distal portion, and a body portion extending between the proximal portion and the distal portion, the body portion defining a lumen, wherein the proximal portion comprises at least one bypass opening extending from an exterior of the introducer sheath to the lumen of the introducer sheath; and
wherein the introducer sheath further includes a plug positioned within the lumen and proximally relative to the at least one bypass opening of the introducer sheath to inhibit the flow of blood into the lumen proximal of the plug.

12. The device delivery system of claim 11, wherein the at least one bypass opening is lined with a radiopaque material.

13. The device delivery system of claim 11, wherein the plug is composed of a radiopaque material.

14. The device delivery system of claim 11, wherein the at least one bypass opening comprises a plurality of bypass openings.

15. The device delivery system of claim 11, wherein the at least one bypass opening comprises a plurality of bypass openings.

16. The device delivery system of claim 11, wherein the plug comprises a central opening configured for receiving a catheter positioned within the introducer sheath.

17. The device delivery system of claim 11, wherein the plug comprises a central opening composed of a slit.

18. A method for delivering a percutaneous device into a patient; comprising:
inserting an introducer sheath into a vessel of the patient, the introducer sheath comprising a proximal portion opposite a distal portion, a body portion extending between the proximal portion and the distal portion and the body portion defining a lumen, and at least one bypass opening arranged at the proximal portion of the introducer sheath;
inserting the percutaneous device into the introducer sheath and advancing the device to a target position in the patient; and
inserting a plug into the introducer sheath arranged such that the plug is positioned adjacent to and proximally relative to the at least one bypass opening to inhibit the flow of blood into the lumen proximal of the plug.

19. The method of claim 18, wherein the method further includes removing the plug from the introducer sheath and removing the percutaneous circulatory support device through the introducer sheath.

20. The method of claim 18, wherein the at least one bypass opening comprises a plurality of bypass openings and the plug is positioned proximally of a proximal-most bypass opening of the plurality of bypass openings.
